(19) United States
(12) Plant Patent Application Publication
(10) Pub. No.: US 2003/0217400 P1
(43) Pub. Date: Nov. 20, 2003

(54) Prunus plant named 'VSL-2'

(76) Inventor: Guennadi Eremin, Krymsk (RU)

Correspondence Address: Ms. Shannon K. Reeser, Varieties International, P.O. Box 753, Lafayette, OR, 97127, US

(21) Appl. No.: 09/880,953

(22) Filed: Jan. 11, 2002

(51) Int. Cl. A01H 5/00

(52) U.S. Cl. PLT/183

(57) ABSTRACT

A new and distinct rootstock variety of Prunus named ‘VSL-2’ that is characterized by an absence of suckers, a vigorous growth habit, pink flowers, the ability to serve well as rootstock that is compatible with all other cherries, and the ability to propagate well by softwood cuttings, stool beds and meristem cuttings in vitro. In combination these traits set the new cultivar apart from all other existing varieties of Prunus rootstock.

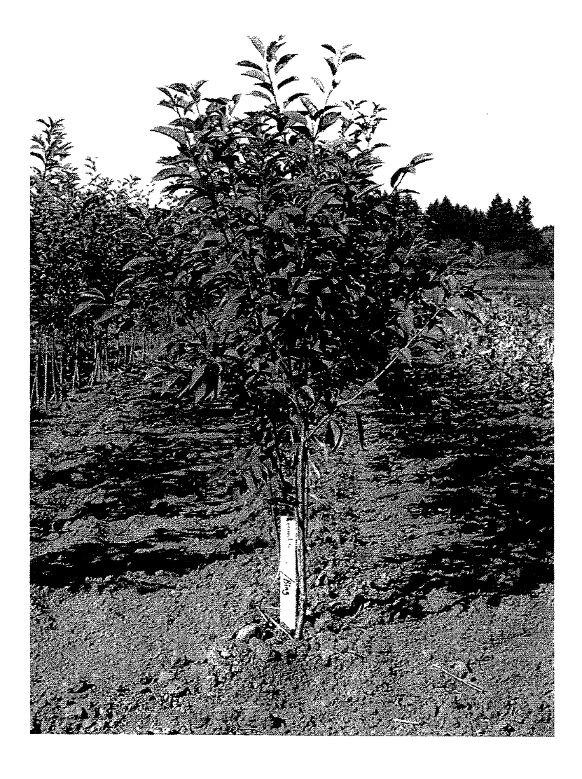

### CROSS-REFERENCES TO RELATED APPLICATIONS

The application for the new invention Prunus Plant Named ‘VSL-2’ will be co-pending with three other applications entitled Prunus Plant Named ‘VVA-1’, Prunus Plant Named ‘LC-52’, and Prunus Plant Named ‘VSV-1’ having the same filing date and inventor.

### BACKGROUND OF THE INVENTION

The present invention relates to the new and distinct cultivar known botanically as a hybrid of Prunus and referred to hereinafter as ‘VSL-2’. The new invention was bred by the inventor at the Breeding Station in Krymsk, Russia.

The breeding program at the Breeding Station was established in 1976 and funded by the government of the former Soviet Union for the purpose of producing new and improved Prunus cultivars that propagate well by methods of softwood cuttings, meristem cuttings in vitro, as well as by stool beds, and that serve well as rootstock that is compatible with other cherries.

In 1976 the inventor crossed the female Prunus fruticosa (not patented) with the male Prunus lannesiana (not patented) producing an induced hybridization in a cultivated area of Krymsk, Russia. The resulting seedlings were observed and evaluated for ten years. In 1986 the inventor selected ‘VSL-2’ from these seedlings. The new cultivar is a hybrid cross between Prunus fruticosa (not patented) and Prunus lannesiana (not patented).

The closest comparison plants are the parent plants. ‘VSL-2’ is distinguishable from the female parent Prunus fruticosa by larger leaves, greater vigor, pink flowers, sour fruit, and absence of suckers. ‘VSL-2’ is distinguishable from the male parent Prunus lannesiana by the absence of stipules, smaller size, dark red fruit, and the ease of propagation. The distinguishing traits that make ‘VSL-2’ unique from all other existing varieties of Prunus are ease of propagation, absence of suckers and the ability to serve as rootstock that is compatible with all cherries.

‘VSL-2’ was first asexually propagated in 1986 by the inventor at the Breeding Station in Krymsk, Russia. The method used was softwood cuttings. The distinguishing traits have been determined stable and are reproduced true to type in successive generations.

### SUMMARY OF THE INVENTION

The following traits have been repeatedly observed and represent the distinguishing characteristics of the new cultivar. These traits in combination distinguish ‘VSL-2’ from all other existing varieties of Prunus. ‘VSL-2’ has not been tested under all possible conditions and phenotypic differences may be observed with variations in environmental, climatic, and cultural conditions, however, without any variance in genotype.

1. ‘VSL-2’ serves as rootstock that is compatible with all other cherries.

2. ‘VSL-2’ propagates well by softwood cuttings, stool beds, and in vitro.

3. ‘VSL-2’ does not produce suckers.

4. ‘VSL-2’ exhibits a dwarf form.

5. ‘VSL-2’ is vigorous.

6. ‘VSL-2’ differs from the female parent Prunus fruticosa by increased vigor, large leaves, pink flowers, sour fruit, and absence of suckers.

7. ‘VSL-2’ differs from the male parent Prunus lannesiana by its smaller size, dark red, sour fruit, ease of propagation, and absence of stipules.

### BOTANICAL DESCRIPTION OF THE PLANT

The following is a detailed botanical description of the new rootstock variety Prunus ‘VSL-2’. Observations, measurements, values, and comparisons were collected in McMinnville, Oreg. from the inventor. The foliage, flower and fruit exhibited by this cultivar are of no economic or commercial value, therefore comparisons and botanical descriptions of the foliage, fruit and flower are made for identification purposes only. Mature specimens, as well as bareroot specimens, were unavailable for photographing at the time this document was written. The color determinations are in accordance with the RHS Colour Chart of The Royal Horticultural Society, London England except where general color terms of ordinary dictionary significance are used.

Botanical classification: Prunus ‘VSL-2’.

Parentage: Prunus ‘VSL-2’ is an induced hybrid that resulted from crossing the following plants.

Female parent.—Prunus fruticosa (not patented).

Male parent.—Prunus lannesiana (not patented).

Type: Deciduous tree.

Use: ‘VSL-2’ serves well as rootstock for all other cherries.

Soil: All soils.

Light: Full sunlight.

Fruit bearing: Low fruit bearing.

Crop time: Requires 3 years from a rooted cutting to achieve finished product size ready to ship bareroot (1 year rootstock and 2 years with scion variey).

Dimensions at crop time: 2.5 m in height and 2 m. in width.

Vigor: 50-60% of standard using Prunus avium as standard (sweet cherry seedling).

Habit: Dwarf and erect.

Hardiness: USDA Zone 5A.

Propagation: Propagation can be accomplished by softwood cuttings, stool beds and meristem cuttings in vitro.

Rooting habit: Fine and fibrous initially. After 1 year roots become fleshy and thick.

Time to initiate roots: 6 months is required to develop roots at 22-25° Centigrade.

Disease and insect resistance: Normal resistance to disease and insects.

Trunk:

Trunk dimensions (at 3 years).—5 cm. in diameter and 20 cm in height above soil.

Trunk bark surface.—Glabrous surface.

Trunk bark color.—178B.

Lenticels.—Present.

Lenticel dimensions.—2-3 mm. in length and 1 mm. in width.

Lenticel shape.—Lens shaped.

Lenticel color.—198B.

Branches:

Branch surface.—Glabrous surface.

Branch color.—175B.

Internode length.—5-10 cm. between nodes.

Branching angle at emergence.—70° angle.

Branching habit.—Freely branching.

Branch pubescence.—Absent.

Branch lenticels.—Present.

Lenticels.—Present on all branches.

Lenticel shape.—Lens shaped.

Lenticel color.—198B.

Lenticel dimensions.—2-3 mm. in length and 1 mm. in width.

Leaves:

Arrangement.—Alternate and whorled.

Leaf length.—4-4.5 cm. in length.

Leaf width.—3-3.5 cm. in width.

Leaf shape.—Lanceolate.

Leaf apex.—Acuminate.

Leaf base.—Rounded.

Leaf color (adaxial surface).—135D.

Leaf color (abaxial surface.—136B.

Leaf surface (adaxial).—Glabrous surface.

Leaf surface (abaxial).—Glabrous surface.

Leaf margins.—Crenulate. Young leaves involuted.

Leaf division.—Simple.

Petiole dimensions.—1.2 cm. in length and 1 mm. in width.

Petiole color.—140C.

Petiole surface.—Glabrous.

Stipules.—Absent.

Leaf pubescence.—Absent.

Venation pattern.—Pinnate with prominent mid-vein.

Vein color (adaxial and abaxial surfaces).—135D.

Leaf texture.—Smooth texture.

Leaf strength.—Moderate strength.

Leaf appearance.—Glossy with young leaves involuted.

Fruit:

Maturity.—Requires 60-65 days to mature.

Dates of picking.—Not a fruit crop. Fruit has no commercial value.

Production.—Minimal

Fruit form.—Globose.

Stem dimensions.—2-2.5 cm. in length and 1 mm. in width.

Stem color.—144A.

Skin color.—53A.

Skin surface.—Glabrous surface.

Lenticels.—Absent.

Flesh color.—53A.

Flesh texture.—Juicy.

Flavor.—Bitter.

Aroma.—None.

Seed number.—One drupe.

Seed shape.—Round.

Seed color.—164A.

Seed dimensions.—5-6 mm. in diameter and 5-6 mm. in length.

Keeping quality.—None.

Storage.—Not determined because the fruit has no commercial value.

Use.—Fruit is not recommended for consumption and has no commercial value.

Flower:

Arrangement.—Solitary.

Flower shape.—Rotate.

Bud dimensions.—3 mm. in width and 3 mm. in length.

Bud color.—56C.

Time of bloom.—Flowers bloom at when plant is 1 year and flowers last for 7-8 days.

Flower diameter.—2.5-3 cm. in diameter.

Flower depth (throat).—2-2.5 mm. in depth.

Bloom quantity.—40-50 flowers per branch.

Number of petals.—Five petals in number.

Fused or unfused.—Petals are unfused.

Petal shape.—Oval.

Petal margin.—Entire and wavy.

Flower color fully opened (upper and lower surfaces).—56D.

Dimensions of peduncle.—2 cm. in length and 1 mm. in width.

Color of peduncle.—144A.

Surface of peduncle.—Glabrous surface.

Calyx dimensions.—2-3 mm. in length and 7-8 mm. in width.

Calyx surface.—Glabrous surface.

Pubescence.—Absent.

Calyx color.—152A.

Number of sepals.—Five sepals in number.

Natural flowering season.—Spring.

Persistent or self-cleaning.—Self-cleaning.

Fragrance.—None.

Reproductive organs:

Stamen number.—Polyandrous. 20-25 stamens and unequal lengths.

Stamen color.—145C.

Anther shape.—Round with stamen attached at center of dorsal surface.

Anther color.—9B.

Anther dimensions.—1 mm. in length and 1 mm. in width.

Amount of pollen.—Heavy amount.

Color of pollen.—9B.

Pistil.—One in number.

Pistil color.—144C.

Pistil dimensions.—1 cm. in length and 0.50 mm. in width.

Style color.—144C.

Style shape.—Elongate.

Style dimensions.—1 mm. in length and 0.50 mm.width.

Ovary dimensions.—2 mm. in length and 2 mm. in width.

Ovary color.—144B.

Ovary position.—Superior.

1. A new and distinct rootstock variety of prunus named ‘VSL-2’ as described herein.
